(19) United States
(12) Patent Application Publication
(10) Pub. No.: US 2024/0221891 A1
(43) Pub. Date: Jul. 4, 2024

(54) A method and system for associating dose related data captured with and stored in an add-on device to a drug delivery device

(71) Applicant: Sanofi, PARIS (FR)

(72) Inventors: Alexander Allerdings, Frankfurt am Main (DE); Adam Moyo Harvey-Cook, Billericay, Essex (GB); David Aubrey Plumptre, Worcestershire (GB); Oliver Charles Gazeley, Basel (CH); Robert Veasey, Warwick (GB); Aiden Michael O`Hare, Warwick (GB)

(21) Appl. No.: 18/570,346

(22) PCT Filed: Jun. 18, 2021
(86) PCT No.: PCT/EP2021/066591

§ 371 Date: Dec. 14, 2023

(51) Int. Cl. G16H 20/17; G06K 7/10; G16H 40/60

(52) U.S. Cl. CPC G16H 20/17; G06K 7/10366; G16H 40/60

(57) ABSTRACT

A method for associating dose related data captured with and stored in an add-on device to a drug delivery device by means of an external computing device is described. The method includes: providing a drug delivery device comprising identification information, providing an add-on device, comprising communication means allowing the add-on device to communicate with an external computing device, means for capturing and storing dose related data, wherein the add-on device is adapted to be releasably mounted on and in engagement with the drug delivery device, providing an external computing device comprising, means for receiving the identification information from the drug delivery device, communication means allowing the external control device to communicate with the add-on device, processor means, and storage means comprising a computer program.

### CROSS REFERENCE TO RELATED APPLICATIONS

The present application is the national stage entry of International Patent Application No. PCT/EP2021/066591, filed on Jun. 18, 2021, the disclosure of which is incorporated herein by reference.

### TECHNICAL FIELD

The present disclosure relates to a method and system for associating dose related data captured with and stored in an add-on device to a drug delivery device, particularly a drug injection device, a drug delivery device being adapted for usage with such a system, an add-on device for a drug delivery device and being adapted for usage such a system, and an external computing device being configured for usage with such a system.

### BACKGROUND

A variety of diseases exists that require regular treatment by delivery, particularly injection of a medicament. Such injection can be performed by using injection devices, which are applied either by medical personnel or by patients themselves.

Drug injection devices particularly for usage by patients themselves may be equipped with electronics for measuring and storing data related to the usage. The usage related data may also be transmitted via a wireless link or a wireline connection to an external device such as a smartphone, a tablet or laptop computer, or in the cloud. For example, US 2019/0134305 A1 discloses a medication delivery device, for example an injection pen or a wearable pump, which can be paired with an external device for providing data captured from a flow sensor relating to medicine delivery to a patient to a paired external device. The device can have Bluetooth® communication and/or near field communication (NFC) circuits for proximity-based pairing and connectivity with the external device for real-time or deferred transfer of captured data to the external device.

Also, add-on modules are known for logging the usage of a drug delivery device, such as the logging device described in EP 3 151 880 B1, which can be releasable attached to a drug delivery device and comprises electronic circuitry comprising sensor means adapted to capture a property value related to a dose amount of drug expelled from a reservoir by the expelling means during an expelling event when the logging device has been attached to a drug delivery device, processor means adapted to determine dose amounts based on captured property values, memory means adapted to store at least one dose amount, display means adapted to display a determined dose amount and/or a time value, and a warning message, and switch means adapted to be operated, when the logging device has been attached to a drug delivery device or is formed integrally therewith, between an off-state when the cap is in the mounted position and an on-state when the cap has been detached. The electronic circuitry may comprise transmitter means adapted to transmit stored data to an external receiver, e.g. by means of NFC or Bluetooth. In order to communicate with the logging module, a smartphone has been provided with specific “insulin diary” software. When the software is activated to initiate data transfer the smartphone NFC transmitter will transmit specific code which will wake up any nearby logging module which will then retransmit a unique code identifying the specific module. If a specific code is received for the first time the user is asked to confirm pairing and is asked to select from a list the given drug that should be associated with the given logging module, e.g. “Mix 30” as shown. Alternatively, the logging module may be designed to work with only one type of pen containing only one specific type of drug, the type of drug being transmitted during initial pairing, or the logging module may be provided with the ability to identify different types of pens and thus different types of drugs.

WO2018/104292A1 relates to medical devices for which the generation, collecting and storing of data are relevant, and more specifically to devices and systems for capturing and organizing drug delivery dose data in a reliable and user-friendly way. A method of pairing a combination of a drug delivery device and an add-on device with an external control device is provided, which comprises the steps of providing a drug delivery device, an add-on device and a control device. The drug delivery device comprises a first visual identifier. The add-on device comprises communication means allowing the add-on device to communicate with an external control device, and a second visual identifier, wherein the add-on device is adapted to be releasable mounted on and in engagement with the drug delivery device, wherein the add-on device is adapted to capture dose related data from a drug delivery device when mounted thereon, and wherein the add-on device is adapted to receive a pairing confirmation from the external control device, whereby the add-on device is actuated from an un-paired to a paired mode. The control device comprises image capturing means, processor means, storage means comprising information in respect of at least one predefined combination of a first and a second visual identifier, and communication means allowing the control device to communicate with the add-on device. The method comprises the further steps of mounting the add-on device on the drug delivery device, capturing an image of the add-on device mounted on the drug delivery device, the image comprising both the first and the second visual identifier, processing the captured image to identify the first and the second visual identifier, and determine if the captured identifiers represent a predefined combination of visual identifiers, and if it is determined that the captured identifiers represent a predefined combination, transmitting a paring confirmation from the control device to the add-on device, thereby actuating the add-on device from the un-paired to the paired mode. One or both of the visual identifiers can be replaced with a NFC tack allowing one or both of the devices to be identified by the NFC functionality of a correspondingly equipped control device.

### SUMMARY

In one aspect, the present disclosure provides a method for associating dose related data captured with and stored in an add-on device to a drug delivery device by means of an external computing device. The method includes the following steps:
providing a drug delivery device comprising identification information,
providing an add-on device, comprising
   communication means allowing the add-on device to communicate with an external computing device,
   means for capturing and storing dose related data from a drug delivery device when mounted thereon,
   wherein the add-on device is adapted to be releasably mounted on and in engagement with the drug delivery device,
providing an external computing device comprising,
   means for receiving the identification information from the drug delivery device, communication means allowing the external control device to communicate with the add-on device,
   processor means,
   storage means comprising a computer program, wherein the computer program configures the external computing device to execute the following steps:
   receiving the identification information from the drug delivery device,
   obtaining information about the drug contained in the drug delivery device based on the identification information, and
labelling captured and stored dose related data based on the received information about the drug.

The method allows using an add-on device, which can be configured for usage with different drug delivery devices for capturing and storing data of dosages expelled with a drug delivery. The dose related data captured by and stored in the add-on device can be associated to the respective drug delivery device with an external computing device, particularly a mobile computing device such as a smartphone, tablet computer, a handheld computer, or laptop computer. Thus, the add-on device does not need any sophisticated hardware for coupling or pairing with a drug delivery device such as a RFID (Radio Frequency Identification) or NFC (Near Field Communication) reader or other means for directly obtaining information from a drug delivery device. Instead, the processing and communication means of an external computing device are used to “couple” the drug delivery device with the add-on device particularly when the external computing device and/or the add-on device establishes a communication connection with each other. The method particularly allows to use an add-on device without any use of an external computing device, particularly without the need for performing a kind of pairing process for the first use. Instead the add-on device can immediately be used, and a user may not require an external computing device for usage such as a smartphone or a mobile computer. An association of dose related data stored in the add-on device with the drug delivery device can only be made when for example the data should be transmitted to an external computing device.

Particularly, the step of providing a drug delivery device comprising identification information may comprise providing a drug delivery device with a RFID tag and/or a visual code, particularly a one- or two-dimensional barcode, comprising the identification information. A RFID tag, particularly a NFC tag, may contain stored data related to the drug delivery device, particularly information on the type of drug contained in the drug delivery device. Such information may be also contained in the visual code. The RFID tag and the visual code stuck, glued, or printed on or integrated in a body of the drug delivery device with relatively less technical means and do not require a dedicated energy source such as a battery. This is particularly useful for dispensable drug injection devices such as non-reusable insulin injection pens usually comprising only a drug cartridge with a syringe and a mechanism for drug expelling, but no electronics and energy source.

The external computing device may comprise one or more of the following: a RFID tag reader; a camera; textual input means; a microphone; and the step of receiving the identification information from the drug delivery device may comprise one or more of the following steps:
reading data from a RFID tag of the drug delivery device with the RFID tag reader and processing the read data to obtain the identification information;
capturing an image of the visual code with the camera and processing the captured image to obtain the identification information;
inputting the visual code via the textual input means and processing the inputted visual code to obtain the identification information;
receiving voice input via the microphone and performing speech processing of the received voice input to obtain the identification information.

Image processing and speech processing for obtaining the identification information require more computing resources, but are comfortable particularly for handicapped users, while the RFID tag reading is also comfortable, but requires an external computing device equipped with a RFID reader. The simplest way for obtaining the identification information are the textual input means, which are however less comfortable than the other measures and may be also more error-prone.

The step of obtaining information about the drug contained in the delivery device based on the identification information may comprise one or more of the following steps: retrieving the information about the drug contained in the drug delivery device from a database stored in the storage means, wherein the database comprises datasets of drugs contained in drug delivery devices and associated identification information; retrieving the information about the drug contained in the drug delivery device from a database stored on an external server via a communication connection between the external computing device and the external server, wherein the database comprises datasets of drugs contained in drug delivery devices and associated identification information;
decoding the information about the drug contained in the drug delivery device from the identification information;
receiving the information about the drug contained in the drug delivery device together with the identification information and/or as part of the identification information. The external computing device may for example comprise the database in its storage so that no further connection to another device is required for obtaining the information on the drug. This might be useful when the database is small enough to be stored for example in the internal storage of a smartphone or tablet computer. However, when the database is too big to be stored in the internal storage of a smartphone or tablet computer, it might be more useful to retrieve the information from the database stored on an external server accessible for example via the internet. A technically less complex approach is to encode information about the drug in the identification information and decode this information with the external computing device, for example by using a barcode software decoder when the identification information is embodied by a one- or two-dimensional barcode. An even simpler solution is directly embedding the drug information in the identification information so that a user can read the drug information. Two or even more of these implementations can be combined particularly to provide redundancy and, thus, increase safety for obtaining the correct drug information.

The step of labelling captured and stored dose related data based on the received information about the drug may comprise at least one of the following:
transmitting the obtained information about the drug from the external computing device to the add-on device and labelling the captured and stored dose related data based on the received information about the drug by the add-on device; transmitting the captured and stored dose related data from the add-on device to the external computing device and labelling the captured and stored dose related data based on the received information about the drug by the external computing device. Thus, labelling may be done by the add-on device and/or by the external computing device.

The method may comprise a step of pairing the external computing device with the add-on device, wherein the transmitting of the obtained information about the drug from the external computing device to the add-on device may be performed during the pairing step and/or the transmitting of the captured and stored dose related data from the add-on device to the external computing device is performed during the pairing step. For example, the information about the drug may be transmitted from the external computing device to the add-on device the first time when the external computing device and the add-on device perform the pairing step and couple the add-on device with the drug delivery device by associating the dose related data stored in the add-on device to the drug delivery device.

The step of paring may require the identification information of the drug delivery device and/or the obtained information about the drug contained in the delivery device in order to complete the pairing of the external computing device with the add-on device. This may be useful to avoid that dose related data stored on the add-on device can be transmitted to a paired external computing device without any information about the drug.

The add-on device may be adapted to switch into a pairing mode when it is attached to the drug delivery device. For example, the add-on device may comprise some switching means for detecting when it is attached to a drug delivery device. The detection of such events by the switching means may cause a switching of the add-on device into the pairing mode so that for example for the first use a pairing of the add-on device with the drug delivery device may be completed to couple the add-on device to the switching device so that dose related data stored in the add-on device may be labelled for associating them with the drug delivery device.

Furthermore, the computer program may configure the external computing device to execute the step of pairing the external computing device with the add-on device during or after the step the receiving the identification information of the drug delivery device. Thus, the computer program may automatically initiate a pairing of the drug delivery device with an add-on device without any user interaction. This may make the usage of the method more comfortable for users since they do not have to take care of changing settings of the external computing devices for pairing.

The step of pairing may comprise a step of erasing dose related data stored in the add-on device and/or paring information from a previous pairing stored in the add-on device.

This may ensure that the add-on device can only be coupled to one drug delivery device and may simplify the management of stored dose related data on the add-on device.

In a further aspect the present disclosure provides a system for associating dose related data captured with and stored in an add-on device to a drug delivery device by means of an external computing device according to a method as disclosed herein, the system comprising a drug delivery device,
an add-on device, and an external computing device, wherein
the drug delivery device comprises an identification information,
the add-on device comprises
   communication means allowing the add-on device to communicate with the external computing device,
   means for capturing and storing dose related data from the drug delivery device when mounted thereon,
   wherein the add-on device is adapted to be releasably mounted on and in engagement with the drug delivery device,
the external computing device comprises
   means for receiving the identification information of the drug delivery device, communication means allowing the external control device to communicate with the add-on device,
   processor means,
   storage means comprising a computer program, wherein the computer program configures the external computing device to
   receive the identification information from the drug delivery device,
   obtain information about the drug contained in the drug delivery device based on the identification information, and
wherein the add-on device and/or external computing device is/are configured to label captured and stored dose related data based on the received information about the drug.

In a et further aspect the present disclosure provides a drug delivery device being adapted for usage with a system as disclosed herein and comprising a RFID tag and/or a visual code, particularly a one- or two-dimensional barcode, comprising the identification information.

In a yet aspect the present disclosure provides an add-on device for a drug delivery device and being adapted for usage with a system as disclosed herein, the add-on device comprising
communication means allowing the add-on device to communicate with an external computing device, and
means for capturing and storing dose related data from a drug delivery device when mounted thereon,
wherein the add-on device is adapted to be releasably mounted on and in engagement with the drug delivery device.

The communication means of the add-on device may comprise one or more of the following: a Bluetooth® communication module; a RFID tag.

In a yet further aspect the present disclosure provides an external computing device being configured for usage with a system as disclosed herein, the external computing device comprising
means for receiving an identification information of a drug delivery device,
communication means allowing the external control device to communicate with an add-on device for the drug delivery device,
processor means,
storage means comprising a computer program, wherein the computer program configures the external computing device to execute the following steps:
receiving the identification information of the drug delivery device,
obtaining information about the drug contained in the delivery device based on the identification information.

### BRIEF DESCRIPTION OF THE FIGURES

FIG. 1 shows embodiments of a drug delivery device comprising identification information.

FIG. 2 shows a block diagram of an embodiment of a system for associating dose related data captured with and stored in an add-on device to a drug delivery device by means of an external computing device.

FIG. 3 shows an embodiment of a system for associating dose related data captured with and stored in an add-on device to a drug delivery device by means of a smartphone applied as an external computing device.

FIG. 4 shows a flowchart of an embodiment of method for associating dose related data captured with and stored in an add-on device to a drug delivery device by means of an external computing device.

### DETAILED DESCRIPTION

In the following, embodiments of the present disclosure will be described with reference to injection devices, particularly an injection device in the form of a pen. The present disclosure is however not limited to such application and may equally well be deployed with other types of drug delivery devices, particularly with another shape than a pen.

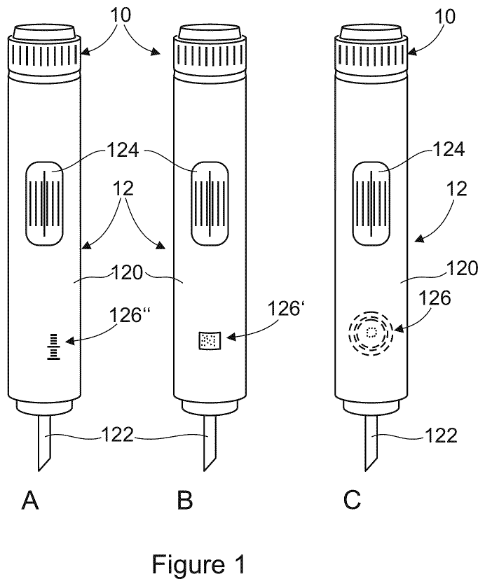

FIG. 1 shows a drug delivery device 12 in the shape of an insulin injection pen. The device 12 comprises an elongated body 120 having a pen-like shaped form for holding a drug cartridge and a dosage selection and delivery mechanism. At the lower end of the body 120, a syringe 122 for expelling a drug dosage and injecting this dosage in a patient's body is provided. The body 120 comprises at its other, upper end a dial knob (not visible) for selecting a drug dosage and an injection knob (not visible) for delivery of a selected dosage. A user of the device 12 selects a dosage by rotating the dial knob around the longitudinal axis of the body 120. The selected dosage is shown on a display 124 integrated in the body 120. After dosage selection, the user may press the injection knob in the direction of the longitudinal axis for expelling the selected dosage via the syringe 122 into a patient's body. A dosage selection and delivery mechanism (not visible) is contained in the body and provided for controlling the dosage selection and expelling. The dosage selection and delivery mechanism may particularly consist only of mechanical parts.

The drug delivery device comprises an identification information provided for obtaining information about the drug contained in the drug delivery device 12, particularly the insulin type contained in an insulin injection pen. The identification information may for example comprise a RFID tag 126, particularly embedded in the body 120 or stuck on the body 120, as shown by the embodiment C in FIG. 1, or a visual code as shown by the embodiments A and B in FIG. 1. In embodiment A, the identification information comprises a one-dimensional barcode 126″, and in the embodiment B, the identification information comprises a two-dimensional barcode 126′ such as a QR code.

The dial know and the injection knob of the drug delivery device 12 shown in FIG. 1 are covered by an add-on device 10, which is adapted to be releasably mounted on and in engagement with the drug delivery device 12, for example clipped on the dial know at the upper end of the body 120. The add-on device 10 may be fixed to the dial knob such that it still allows a rotation of the dial knob, and enables pressing of the injection knob to expel a selected dosage.

The add-on device 10 is particularly a reusable module and may be provided for attachment to suitably configured injection pens for the purpose of recording doses that are delivery from the pens. Injection pens may be configured to deliver a variety of drugs at different dose rates, for example a number of insulin units per click of a dosage selection mechanism. The add-on device 10 therefore comprises means for capturing dose related data, for example optical detector means or other sensor means to detect a selected and expelled dosage.

The capturing and storing of dose related data with the add-on device 10 may be performed only when the add-on device 10 mounted on and in engagement with the drug delivery device 12. For example, the add-on device 10 may comprise a switch (not shown), which is activated upon engagement of the add-on device 10 with the drug delivery device 12. The activation of the switch may for example power up an add-on device internal electronics for capturing and storing dose related data.

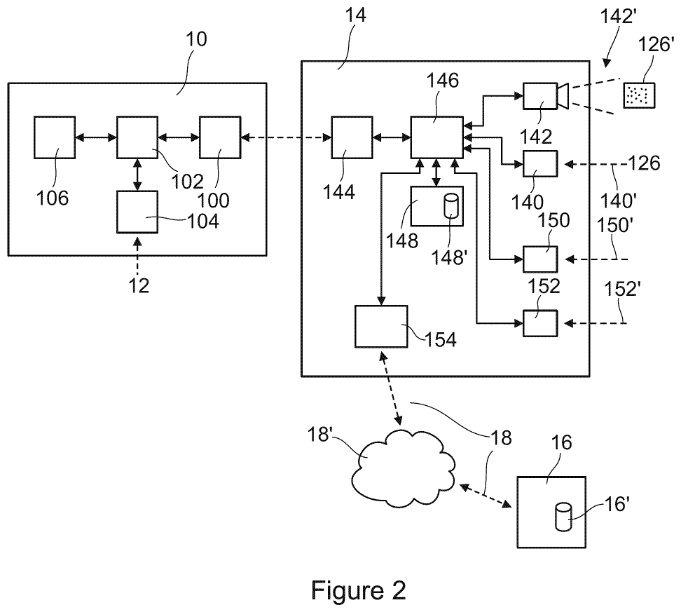

The add-on device 10 may be provided for usage with different drug delivery devices 12, and, thus, an association of dose captured with and stored in the add-on device 10 to a drug delivery 12 by means of an external computing device is provided. The associating will be now be explained with reference to the block diagram of a system shown in FIG. 2.

The add-on device 10 comprises detector means 104 for capturing a dosage selected and expelled with a drug delivery device 12, on which the add-on device 10 is mounted and with which it is in engagement. As mentioned above, the detector means 104 may comprise optical detector means. For example, the add-on device may comprise one or more LEDs (Light Emitting Diodes) and one or more photodiodes to detect reflections of radiation emitted by the one or more LEDs from optical encoding means of a drug selection mechanism of the drug delivery device 12 via the one or more photodiodes. When a dosage is selected by a rotation of the dial knob, also the optical encoding means may be rotated and, thus, a specific reflection pattern related to the selected dosage may be generated by the rotation. This specific reflection patterns may be detected by the one or more photodiodes and converted into an electrical signal representing a selected dosage. A click detector of the add-on device 10 may then detect a pressing of the injection knob and expelling of the selected dosage. The captured selected and expelled dosage may then be stored in an internal memory of the add-on device in a digital form as dose related data.

The add-on device 10 further comprises a controller 102 for controlling the operation of the add-on device 10. The controller 102 may be for example a microcontroller configured with a dedicated firmware to perform the required function to process captured selected and expelled dosages in an internal storage 106 of the add-on device 10.

Furthermore, the add-on device 10 comprises communication means 100, for example a wireless communication module for wirelessly communicating via the Bluetooth® standard, the WiFi™ standard or via a RFID standard, particularly a NFC communication standard. The communication means 100 are connected to the controller 102 and particularly provided to transmitting data stored in the internal storage 106 to an external computing device 14 and/or for receiving data from the external computing device 14 for further processing with the controller 102, as will be described later in more detail.

The electronics of the add-on device 10 comprising the means 100 to 106 may only be powered when the add-on device 10 is mounted on a drug delivery device 12 and engaged with it, and when for example a dosage is selected by rotating the add-on device 10 clipped on a dial know for dosage selection of the drug delivery device 12. A button cell, for example of type CR2032, may be provided as power supply for the electronics of the add-on device 10.

Selected and expelled dosages are captured by the electronics of the add-on device 10 and internally stored in the storage 106 of the add-on device 10 as dose related data. The dose related data may comprise for each captured selected and expelled dosage a dedicated dataset, which may also comprise a date and time stamp.

The stored dose related data can be associated with the drug contained in the drug delivery device 12 by means of the external computing device 14, which may be for example a smartphone, a tablet, laptop or desktop computer, or a smartwatch.

The external computing device 14 comprises means 140, 142, 150, 152 for receiving the identification information 126, 126′ from the drug delivery device 12 (in the following generally designated as identification information receiver means).

The identification information receiver means may comprise various implementations. For example, they may comprise a RFID reader, particularly NFC reader 140 for reading data 140′ from a RFID or NFC tag 126 of the drug delivery device 12. The identification information receiver means may also comprise a camera 142 for capturing an image 142′ of a visual code, for example a two-dimensional barcode 126′, printed on the body 120 of the drug delivery device 12. In another implementation, the identification information receiver means may comprise textual input means 150 such as a keyboard for receiving a textual input 150′ of the visual code comprising the identification information entered manually by a patient. The identification information receiver means may in yet another implementation comprise a microphone 152 for receiving a voice input 152′ form a patient, wherein the voice input 152′ may comprise spoken phrases comprising the identification information.

The external computing device 14 comprises processor means 146 such as a processor or microcontroller, which are configured by a computer program, for example an app downloaded to and installed on the external computing device 14 for configuring the external computing device 14 to associate dose related data captured with and stored the the add-on device 10 to the drug delivery device 12.

The computer program is stored in internal storage means 148 of the external computing device 14, for example a flash memory of a smartphone. The storage means 148 may also comprise a database 148′ containing information about drugs, particularly associations of identification information of drug delivery devices associated with drugs.

The external computing device 14 comprises communication means 144, for example a wireless communication module for wirelessly communicating via the Bluetooth® standard, the WiFi™ standard or via a RFID standard, particularly a NFC communication standard. The communication means 144 are particularly provided to receiving data from the add-on device 10, which are stored in its storage 106.

The external computing device 14 may further comprise communication means 154 for establishing a communication connection 18 over a wide area network (WAN), for example the internet 18′, with an external server 16 hosting a database 16′ storing information about drugs stored in drug delivery devices. The database 16′ may be for example hosted by a manufacturer of drug delivery devices for identifying drugs contained in the devices. The database 16′ may for example contain codes of drug delivery devices produced by a manufacturer and drugs assigned to these codes in order to enable a retrieval of drug delivery devices and their drug contents.

The database 16′ may comprise the database 148′ and particularly a more comprehensive database, i.e. contain a superset of information on drugs contained in drug delivery devices of a manufacturer. For example, database 16′ may contain information on all produced drug delivery devices, while database 148′ may contain only a subset of these devices, for example onla information on a certain kind of drug delivery devices.

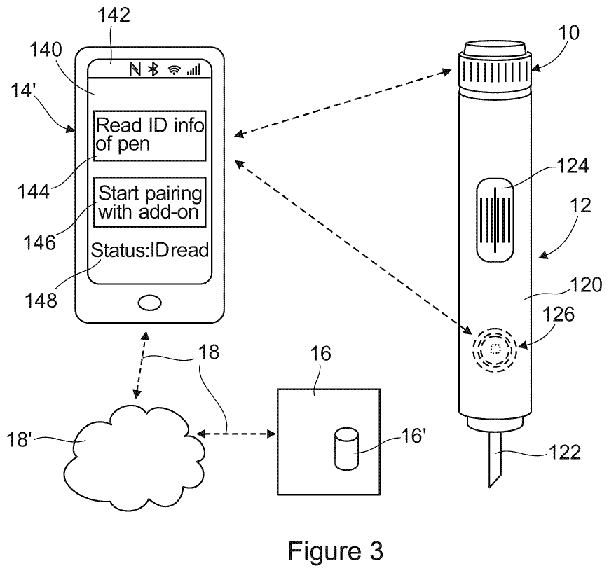

FIG. 3 shows an implementation of the system of FIG. 2 with a smartphone 14′ as external computing device. The smartphone 14′ may be equipped with at least one of the following: a camera; a NCF reader; a Bluetooth® communication module. The status bar 142 displayed at the top of the display 140 of the smartphone 14′ shows activated communication means of the smartphone such as NFC, Bluetooth®, WiFi™, Cellular radio. An app is installed on the smartphone 14′, which configures it to perform a method for associating dose related data captured with and stored in the add-on device 10 to the drug delivery device 12. The app shows on the display 140 different touch buttons 144, 146 for performing actions and a status message 146.

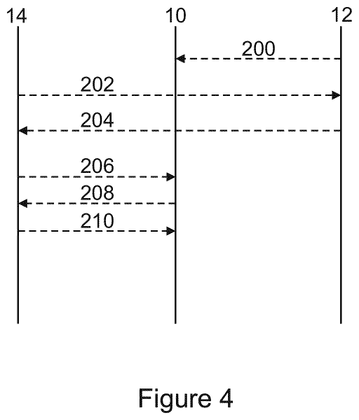

Now, the operation of the system shown in FIG. 3 is explained with reference to the flowchart shown in FIG. 4. When the add-on device 10 is attached to and engaged with the drug delivery device 12, selection and expelling of dosages with the drug delivery device 12 are detected and internally stored by the add-on device 10 (arrow 200 in FIG. 4). This does not require any kind of pairing of the add-on device 10 with the drug delivery device 12 and/or the smartphone 14′. Thus, the internally stored dose related data are after the first use not associated with the drug delivery device 12, and particularly the drug contained in the drug delivery device 12. Thus, usage of the add-on device 10 with different drug delivery devices 12 is easily possible and does not require any dedicated hard- and/or software for first use.

For associating the dose related data stored in the add-on device 10, the app, which is particularly provided for association of the add-on device 10 and the drug delivery device 12, is launched on the smartphone 14′. A user may then touch the button 144 for receiving the identification information from the drug delivery device 12.

The identification information may be received for example via NFC by activating a NFC reader of the smartphone 14′ for reading identification information of the drug delivery device 12 stored in the NFC tag 126, by powering the passive NFC tag 126 (arrow 202 in FIG. 4), which in response transmits data stored in an internal storage of the NFC tag 126 to the smartphone 14′ (arrow 204 in FIG. 4).

The identification information may also be received for example by taking a picture of a barcode printed on the body 120 of the drug delivery device 12 (refer to FIGS. 1A and B) and processing the captured image of the barcode to obtain the identification information.

A further possibility of receiving the identification information may be by inputting a visual code for example printed on the drug delivery device or on a leaflet accompanying the drug delivery device via textual input means such as a software generated keyboard shown in the display 140 of the smartphone 14′, or by speech processing of a voice input received via the microphone of the smartphone 14′.

The app may then retrieve information about the drug contained in the drug delivery device 12 based on the received identification information for example from the internal database 148′ stored in the smartphone storage 148 or from the external database 16′ hosted by the external server 16 via the WAN connection 18 established with the communication means 154. Retrieved drug information can be for example shown with a status message on the display 140. The drug information may be also contained in the received identification information, for example in an encoded form or as part of the identification information.

Next, a user may initiate a pairing step or process of the smartphone 14′ with the add-on device 10 by touching button 146 shown on the smartphone display 140 by the app. The pairing process may also be automatically initiated without any user interaction after the reading of the identification information from the drug delivery device 12. The completion of the pairing process may require the identification information from the drug delivery device 12 and/or the obtained information about the drug. The pairing process may be for example not completed by the app when no identification information and/or no drug information is available, for example because no identification information was received from the drug delivery device 12 and/or no information on the drug could be retrieved.

The pairing process may comprise activating the Bluetooth® module of the smartphone 14′ and sending a communication request to the add-on device 10 (arrow 206 in FIG. 4), which in response establishes a Bluetooth® communication connection with the smartphone 14′ (arrow 208 in FIG. 4). After establishing the Bluetooth® communication connection, the smartphone 14′ may then transmit the drug information previously retrieved by the smartphone 14′ to the add-on device 10 (arrow 210 in FIG. 4), which then is used by the add-on device 10 to label the internally stored dose related data with the received drug information.

The pairing process may alternatively comprise activating a NFC reader-writer module of the smartphone 14′ and sending a communication request to the add-on device 10 containing the drug information (arrow 206 in FIG. 4). A NFC circuitry of the add-on device 10 may be powered by the electromagnetic field generated by the NFC reader-writer module of the smartphone 14′. The drug information previously retrieved by the smartphone 14′ may be transmitted to the add-on device 10 with a further write request. The NFC circuitry of the add-on device 10 may in response to this label internally stored dose related data with the received drug information. This may be acknowledged with a respective reply message transmitted in response to the received write request.

Alternatively or additionally, the labelling may be also performed by the smartphone 14′, which requires that the stored dose related data is transmitted from the add-on device 10 to the smartphone 14′ after establishing the a communication connection between the add-on device 10 and the smartphone 14′. The smartphone 14′ can after receipt of the captured dose related data from the add-on device 10 label these data, and either store them internally and/or transmit the labelled data back to the add-on device 10, which may then store the received and labelled data by overwriting the previously transmitted unlabelled stored dose related data.

The labelling by the add-on device 10 may comprise storing the received drug information in the internal storage of the add-on device 10 together with the dose related data. Thus, the dose related data stored in the add-on device 10 is associated with the drug delivery device 10. The completed labelling can be displayed with a respective status message on the display 140 of the smartphone 14′. The labelling may only be required during or for the pairing process. Later, when dose related data should be transmitted from the add-on device 10 to the smartphone 14′, for example for displaying them on the smartphone display 140 or storing them in an account of a user for example on the external server 16, a further pairing process may not be required since the dose related data stored in the add-on device 10 has already been labelled during the previous pairing process.

The add-on device 10 may also be adapted to be switched in a pairing mode upon attachment to and/or detachment from the drug delivery device 12. Thus, any change of the add-on device 10 with different drug delivery devices may cause a switching into the pairing mode of the add-on device 10 so that a smartphone can recognize when the drug delivery device has been changes. This may also reduce the energy consumption of an add-on device 10, which is for example equipped with a Bluetooth® module. The Bluetooth® module may be powered only upon attachment or detachment. This may be for example accomplished by some switching means integrated in the add-on device 10, which may be activated when the add-on device 10 is attached, and once the switching means are activated, the Bluetooth® module may be powered for a certain time and switched into the pairing mode in which the labelling of internally stored dose related data may be accomplished by means of an external computing device such as the smartphone 14′.

The above described pairing can already been executed during the receiving of the identification information of the drug delivery device 12. For example, the pairing may be initiated once the smartphone 14′ is configured to read the identification information from the NFC tag 126. This means that for example the Bluetooth® module or NFC reader-writer module of the add-on device 10 may be already activated during the receiving of the identification information.

Finally, the pairing may also comprise a step of erasing of dose related data stored in the add-on device, for example from a previous usage of the add-on device with another drug delivery device. Thus, a pairing comprises a kind of resetting the add-on device for preparation for a new usage with a new drug delivery device. Alternatively or additionally, a pairing stored in the add-on device may be erased by a new pairing. These erasing may be only used with add-on devices, which are provided for usage with a single drug delivery device. When the add-on device should be used with several drug delivery devices, it may be configured to store several pairings and dose related data captured from different drug delivery devices, and separately store these different dose related data.

The terms “drug” or “medicament” are used synonymously herein and describe a pharmaceutical formulation containing one or more active pharmaceutical ingredients or pharmaceutically acceptable salts or solvates thereof, and optionally a pharmaceutically acceptable carrier. An active pharmaceutical ingredient (“API”), in the broadest terms, is a chemical structure that has a biological effect on humans or animals. In pharmacology, a drug or medicament is used in the treatment, cure, prevention, or diagnosis of disease or used to otherwise enhance physical or mental well-being. A drug or medicament may be used for a limited duration, or on a regular basis for chronic disorders.

As described below, a drug or medicament can include at least one API, or combinations thereof, in various types of formulations, for the treatment of one or more diseases. Examples of API may include small molecules having a molecular weight of 500 Da or less; polypeptides, peptides and proteins (e.g., hormones, growth factors, antibodies, antibody fragments, and enzymes); carbohydrates and polysaccharides; and nucleic acids, double or single stranded DNA (including naked and cDNA), RNA, antisense nucleic acids such as antisense DNA and RNA, small interfering RNA (siRNA), ribozymes, genes, and oligonucleotides. Nucleic acids may be incorporated into molecular delivery systems such as vectors, plasmids, or liposomes. Mixtures of one or more drugs are also contemplated.

The drug or medicament may be contained in a primary package or “drug container” adapted for use with a drug delivery device. The drug container may be, e.g., a cartridge, syringe, reservoir, or other solid or flexible vessel configured to provide a suitable chamber for storage (e.g., short- or long-term storage) of one or more drugs. For example, in some instances, the chamber may be designed to store a drug for at least one day (e.g., 1 to at least 30 days). In some instances, the chamber may be designed to store a drug for about 1 month to about 2 years. Storage may occur at room temperature (e.g., about 20° C.), or refrigerated temperatures (e.g., from about −4° C. to about 4° C.). In some instances, the drug container may be or may include a dual-chamber cartridge configured to store two or more components of the pharmaceutical formulation to-be-administered (e.g., an API and a diluent, or two different drugs) separately, one in each chamber. In such instances, the two chambers of the dual-chamber cartridge may be configured to allow mixing between the two or more components prior to and/or during dispensing into the human or animal body. For example, the two chambers may be configured such that they are in fluid communication with each other (e.g., by way of a conduit between the two chambers) and allow mixing of the two components when desired by a user prior to dispensing. Alternatively, or in addition, the two chambers may be configured to allow mixing as the components are being dispensed into the human or animal body.

The drugs or medicaments contained in the drug delivery devices as described herein can be used for the treatment and/or prophylaxis of many different types of medical disorders. Examples of disorders include, e.g., diabetes mellitus or complications associated with diabetes mellitus such as diabetic retinopathy, thromboembolism disorders such as deep vein or pulmonary thromboembolism. Further examples of disorders are acute coronary syndrome (ACS), angina, myocardial infarction, cancer, macular degeneration, inflammation, hay fever, atherosclerosis and/or rheumatoid arthritis. Examples of APIs and drugs are those as described in handbooks such as Rote Liste 2014, for example, without limitation, main groups 12 (anti-diabetic drugs) or 86 (oncology drugs), and Merck Index, 15th edition.

Examples of APIs for the treatment and/or prophylaxis of type 1 or type 2 diabetes mellitus or complications associated with type 1 or type 2 diabetes mellitus include an insulin, e.g., human insulin, or a human insulin analogue or derivative, a glucagon-like peptide (GLP-1), GLP-1 analogues or GLP-1 receptor agonists, or an analogue or derivative thereof, a dipeptidyl peptidase-4 (DPP4) inhibitor, or a pharmaceutically acceptable salt or solvate thereof, or any mixture thereof. As used herein, the terms “analogue” and “derivative” refers to a polypeptide which has a molecular structure which formally can be derived from the structure of a naturally occurring peptide, for example that of human insulin, by deleting and/or exchanging at least one amino acid residue occurring in the naturally occurring peptide and/or by adding at least one amino acid residue. The added and/or exchanged amino acid residue can either be codable amino acid residues or other naturally occurring residues or purely synthetic amino acid residues. Insulin analogues are also referred to as “insulin receptor ligands”. In particular, the term “derivative” refers to a polypeptide which has a molecular structure which formally can be derived from the structure of a naturally occurring peptide, for example that of human insulin, in which one or more organic substituent (e.g. a fatty acid) is bound to one or more of the amino acids. Optionally, one or more amino acids occurring in the naturally occurring peptide may have been deleted and/or replaced by other amino acids, including non-codeable amino acids, or amino acids, including non-codeable, have been added to the naturally occurring peptide.

Examples of insulin analogues are Gly(A21), Arg(B31), Arg(B32) human insulin (insulin glargine); Lys(B3), Glu(B29) human insulin (insulin glulisine); Lys(B28), Pro(B29) human insulin (insulin lispro); Asp(B28) human insulin (insulin aspart); human insulin, wherein proline in position B28 is replaced by Asp, Lys, Leu, Val or Ala and wherein in position B29 Lys may be replaced by Pro; Ala(B26) human insulin; Des(B28-B30) human insulin; Des(B27) human insulin and Des(B30) human insulin.

Examples of insulin derivatives are, for example, B29-N-myristoyl-des(B30) human insulin, Lys(B29) (N-tetradecanoyl)-des(B30) human insulin (insulin detemir, Levemir®); B29-N-palmitoyl-des(B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl-ThrB29LysB30 human insulin; B29-N—(N-palmitoyl-gamma-glutamyl)-des(B30) human insulin, B29-N-omega-carboxypentadecanoyl-gamma-L-glutamyl-des(B30) human insulin (insulin degludec, Tresiba®); B29-N—(N-lithocholyl-gamma-glutamyl)-des(B30) human insulin; B29-N-(ω-carboxyheptadecanoyl)-des(B30) human insulin and B29-N-(ω-carboxyheptadecanoyl) human insulin.

Examples of GLP-1, GLP-1 analogues and GLP-1 receptor agonists are, for example, Lixisenatide (Lyxumia®), Exenatide (Exendin-4, Byetta®, Bydureon®, a 39 amino acid peptide which is produced by the salivary glands of the Gila monster), Liraglutide (Victoza®), Semaglutide, Taspoglutide, Albiglutide (Syncria®), Dulaglutide (Trulicity®), rExendin-4, CJC-1134-PC, PB-1023, TTP-054, Langlenatide/HM-11260C, CM-3, GLP-1 Eligen, ORMD-0901, NN-9924, NN-9926, NN-9927, Nodexen, Viador-GLP-1, CVX-096, ZYOG-1, ZYD-1, GSK-2374697, DA-3091, MAR-701, MAR709, ZP-2929, ZP-3022, TT-401, BHM-034. MOD-6030, CAM-2036, DA-15864, ARI-2651, ARI-2255, Exenatide-XTEN and Glucagon-Xten.

An examples of an oligonucleotide is, for example: mipomersen sodium (Kynamro®), a cholesterol-reducing antisense therapeutic for the treatment of familial hypercholesterolemia.

Examples of DPP4 inhibitors are Vildagliptin, Sitagliptin, Denagliptin, Saxagliptin, Berberine.

Examples of hormones include hypophysis hormones or hypothalamus hormones or regulatory active peptides and their antagonists, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, and Goserelin.

Examples of polysaccharides include a glucosaminoglycane, a hyaluronic acid, a heparin, a low molecular weight heparin or an ultra-low molecular weight heparin or a derivative thereof, or a sulphated polysaccharide, e.g. a poly-sulphated form of the above-mentioned polysaccharides, and/or a pharmaceutically acceptable salt thereof. An example of a pharmaceutically acceptable salt of a poly-sulphated low molecular weight heparin is enoxaparin sodium. An example of a hyaluronic acid derivative is Hylan G-F 20 (Synvisc®), a sodium hyaluronate.

The term “antibody”, as used herein, refers to an immunoglobulin molecule or an antigen-binding portion thereof. Examples of antigen-binding portions of immunoglobulin molecules include F(ab) and F(ab′)2 fragments, which retain the ability to bind antigen. The antibody can be polyclonal, monoclonal, recombinant, chimeric, de-immunized or humanized, fully human, non-human, (e.g., murine), or single chain antibody. In some embodiments, the antibody has effector function and can fix complement. In some embodiments, the antibody has reduced or no ability to bind an Fc receptor. For example, the antibody can be an isotype or subtype, an antibody fragment or mutant, which does not support binding to an Fc receptor, e.g., it has a mutagenized or deleted Fc receptor binding region. The term antibody also includes an antigen-binding molecule based on tetravalent bispecific tandem immunoglobulins (TBTI) and/or a dual variable region antibody-like binding protein having cross-over binding region orientation (CODV).

The terms “fragment” or “antibody fragment” refer to a polypeptide derived from an antibody polypeptide molecule (e.g., an antibody heavy and/or light chain polypeptide) that does not comprise a full-length antibody polypeptide, but that still comprises at least a portion of a full-length antibody polypeptide that is capable of binding to an antigen. Antibody fragments can comprise a cleaved portion of a full length antibody polypeptide, although the term is not limited to such cleaved fragments. Antibody fragments that are useful in the present invention include, for example, Fab fragments, F(ab′)2 fragments, scFv (single-chain Fv) fragments, linear antibodies, monospecific or multispecific antibody fragments such as bispecific, trispecific, tetraspecific and multispecific antibodies (e.g., diabodies, triabodies, tetrabodies), monovalent or multivalent antibody fragments such as bivalent, trivalent, tetravalent and multivalent antibodies, minibodies, chelating recombinant antibodies, tribodies or bibodies, intrabodies, nanobodies, small modular immunopharmaceuticals (SMIP), binding-domain immunoglobulin fusion proteins, camelized antibodies, and VHH containing antibodies. Additional examples of antigen-binding antibody fragments are known in the art.

The terms “Complementarity-determining region” or “CDR” refer to short polypeptide sequences within the variable region of both heavy and light chain polypeptides that are primarily responsible for mediating specific antigen recognition. The term “framework region” refers to amino acid sequences within the variable region of both heavy and light chain polypeptides that are not CDR sequences, and are primarily responsible for maintaining correct positioning of the CDR sequences to permit antigen binding. Although the framework regions themselves typically do not directly participate in antigen binding, as is known in the art, certain residues within the framework regions of certain antibodies can directly participate in antigen binding or can affect the ability of one or more amino acids in CDRs to interact with antigen.

Examples of antibodies are anti PCSK-9 mAb (e.g., Alirocumab), anti IL-6 mAb (e.g., Sarilumab), and anti IL-4 mAb (e.g., Dupilumab).

Pharmaceutically acceptable salts of any API described herein are also contemplated for use in a drug or medicament in a drug delivery device. Pharmaceutically acceptable salts are for example acid addition salts and basic salts.

Those of skill in the art will understand that modifications (additions and/or removals) of various components of the APIs, formulations, apparatuses, methods, systems and embodiments described herein may be made without departing from the full scope and spirit of the present invention, which encompass such modifications and any and all equivalents thereof.

1.-15. (canceled)

16. A method for associating dose related data captured with and stored in an add-on device to a drug delivery device by an external computing device, the method comprising:
providing a drug delivery device comprising identification information,
providing an add-on device, comprising:
a first communication component allowing the add-on device to communicate with an external computing device,
a second component for capturing and storing dose related data from a drug delivery device when mounted thereon,
wherein the add-on device is adapted to be releasably mounted on and in engagement with the drug delivery device, and
providing an external computing device comprising:
a third component for receiving the identification information from the drug delivery device,
an external communication component allowing the external computing device to communicate with the add-on device,
at least one processor, and
a storage component comprising a computer program, wherein the computer program configures the external computing device to execute the following steps:
receiving the identification information from the drug delivery device,
obtaining information about the drug contained in the drug delivery device based on the identification information, and
labelling the captured and stored dose related data based on the received information about the drug.

17. The method of claim 16, wherein providing the drug delivery device comprising identification information comprises providing the drug delivery device with an RFID tag and/or a visual code comprising the identification information.

18. The method of claim 16, wherein the external computing device comprises at least one of the following:
a RFID tag reader;
a camera;
textual input means; or
a microphone,
wherein receiving the identification information from the drug delivery device comprises one or more of the following steps:
reading data from an RFID tag of the drug delivery device with the RFID tag reader and processing the read data to obtain the identification information;
capturing an image of the visual code with the camera and processing the captured image to obtain the identification information;
inputting the visual code via the textual input means and processing the inputted visual code to obtain the identification information;
receiving voice input via the microphone and performing speech processing of the received voice input to obtain the identification information.

19. The method of claim 16, wherein obtaining information about the drug contained in the delivery device based on the identification information comprises one or more of the following steps:
retrieving the information about the drug contained in the drug delivery device from a database stored in the storage means, wherein the database comprises datasets of drugs contained in drug delivery devices and associated identification information;
retrieving the information about the drug contained in the drug delivery device from a database stored on an external server via a communication connection between the external computing device and the external server, wherein the database comprises datasets of drugs contained in drug delivery devices and associated identification information;
decoding the information about the drug contained in the drug delivery device from the identification information;
receiving the information about the drug contained in the drug delivery device together with the identification information and/or as part of the identification information.

20. The method of claim 16, wherein labelling captured and stored dose related data based on the received information about the drug comprises at least one of the following:
transmitting the obtained information about the drug from the external computing device to the add-on device and labelling the captured and stored dose related data based on the received information about the drug by the add-on device; and
transmitting the captured and stored dose related data from the add-on device to the external computing device and labelling the captured and stored dose related data based on the received information about the drug by the external computing device.

21. The method of claim 20, comprising pairing the external computing device with the add-on device, wherein the transmitting of the obtained information about the drug from the external computing device to the add-on device is performed during the pairing step and/or the transmitting of the captured and stored dose related data from the add-on device to the external computing device is performed during the pairing step.

22. The method of claim 21, wherein pairing the external computing device with the add-on device requires the identification information of the drug delivery device and/or the obtained information about the drug contained in the drug delivery device in order to complete the pairing of the external computing device with the add-on device.

23. The method of claim 21, wherein the add-on device is adapted to switch into a pairing mode when it is attached to the drug delivery device.

24. The method of claim 21, wherein the computer program configures the external computing device to execute the step of pairing the external computing device with the add-on device during or after the step of the receiving of the identification information of the drug delivery device.

25. The method of claim 21, wherein pairing the external computing device with the add-on device comprises erasing dose related data stored in the add-on device and/or paring information from a previous pairing stored in the add-on device.

26. A system for associating dose related data captured with and stored in an add-on device to a drug delivery device by means of an external computing device according to a method of any preceding claim, the system comprising:
a drug delivery device;
an add-on device; and
an external computing device,
wherein the drug delivery device comprises an identification information,
wherein the add-on device comprises:
a first communication component allowing the add-on device to communicate with the external computing device, and
a second component for capturing and storing dose related data from the drug delivery device when mounted thereon,
wherein the add-on device is adapted to be releasably mounted on and in engagement with the drug delivery device,
wherein the external computing device comprises:
a third component for receiving the identification information of the drug delivery device,
an external communication component allowing the external computing device to communicate with the add-on device,
at least one processor, and
a storage component comprising a computer program, wherein the computer program configures the external computing device to:
receive the identification information from the drug delivery device,
obtain information about the drug contained in the drug delivery device based on the identification information, and
wherein at least one of the add-on device or the external computing device is configured to label the captured and stored dose related data based on the received information about the drug.

27. The system of claim 26, wherein the drug delivery device comprises an RFID tag and/or a visual code comprising the identification information.

28. The system of claim 26, wherein the add-on device comprises:
a first communication component allowing the add-on device to communicate with an external computing device, and
a second component for capturing and storing dose related data from a drug delivery device when mounted thereon,
wherein the add-on device is adapted to be releasably mounted on and in engagement with the drug delivery device.

29. The system of claim 28, wherein the first communication component comprises one or more of the following:
a Bluetooth® communication module; or
a RFID tag.

30. The system of claim 26, wherein the external computing device comprises:
a third component for receiving an identification information of the drug delivery device,
an external communication component allowing the external control device to communicate with an add-on device for the drug delivery device,
at least one processor, and
a storage component comprising a computer program, wherein the computer program configures the external computing device to execute the following steps:
receiving the identification information of the drug delivery device,
obtaining information about the drug contained in the drug delivery device based on the identification information.

31. The system of claim 26, wherein obtaining information about the drug contained in the delivery device based on the identification information comprises one or more of the following steps:
retrieving the information about the drug contained in the drug delivery device from a database stored in the storage means, wherein the database comprises datasets of drugs contained in drug delivery devices and associated identification information;
retrieving the information about the drug contained in the drug delivery device from a database stored on an external server via a communication connection between the external computing device and the external server, wherein the database comprises datasets of drugs contained in drug delivery devices and associated identification information;
decoding the information about the drug contained in the drug delivery device from the identification information; or
receiving the information about the drug contained in the drug delivery device together with the identification information and/or as part of the identification information.

32. The system of claim 26, wherein labelling the captured and stored dose related data based on the received information about the drug comprises at least one of the following:
transmitting the obtained information about the drug from the external computing device to the add-on device and labelling the captured and stored dose related data based on the received information about the drug by the add-on device; and
transmitting the captured and stored dose related data from the add-on device to the external computing device and labelling the captured and stored dose related data based on the received information about the drug by the external computing device.

33. The system of claim 26, wherein the add-on device is adapted to switch into a pairing mode when it is attached to the drug delivery device.

34. The system of claim 26, wherein the computer program configures the external computing device to pair the external computing device with the add-on device during or after receiving the identification information of the drug delivery device.

35. The system of claim 34, wherein pairing the external computing device with the add-on device comprises erasing the dose related data stored in the add-on device and/or paring information from a previous pairing stored in the add-on device.
